(19) United States
(12) Patent Application Publication
(10) Pub. No.: US 2009/0030086 A1
(43) Pub. Date: Jan. 29, 2009

(54) ANTIMICROBIAL AGENTS

(76) Inventors: Elizabeth Anne Eady, West Yorkshire (GB); Jonathan Howard Cove, West Yorkshire (GB); Daniel James Fitzgerald, Leeds (GB); Scott Seville, West Yorkshire (GB)

Correspondence Address: Weaver Austin Villeneuve & Sampson LLP, P.O. BOX 70250, OAKLAND, CA, 94612-0250, US

(21) Appl. No.: 11/909,072

(22) PCT Filed: Mar. 23, 2006
(86) PCT No.: PCT/GB2006/001077

§ 371 Date: May 27, 2008

(30) Foreign Application Priority Data
Mar. 23, 2005 (GB) 0505909.2

(51) Int. Cl. A61K 31/122; A61P 31/04

(52) U.S. Cl. 514/682; 514/688; 514/689

(57) ABSTRACT

A benzoquinone or hydroquinone for use in the topical treatment of a condition which is caused, exacerbated or transmitted by bacterial, in particular staphylococcal or propionibacterial, activity. The condition may be a skin or skin structure condition such as acne. The invention also provides the use of a benzo- or hydroquinone in the manufacture of a medicament for the treatment of such a condition.

### FIELD OF THE INVENTION

This invention relates to the use of certain compounds as antimicrobial, in particular antibacterial, agents:

### BACKGROUND TO THE INVENTION

Many skin and skin structure conditions are either caused by or exacerbated by bacterial activity. Wound infections are examples, as are acne, atopic eczema, ulcers, folliculitis, mycoses and other primary and secondary skin and skin structure infections.

Staphylococcal infections can also be caused by bacteria present on the skin or on other epithelia such as in the nares or ears. Some of these bacteria are resistant to commonly used antibiotics, rendering their treatment and prevention extremely problematic—the methicillin-resistant S. aureus (MRSA) strains are examples which are currently causing concern, particularly in hospitals, because of the difficulty of finding appropriate treatments.

Acne is an example of a skin condition which affects many people and yet can be extremely difficult to treat effectively. It is a multifactorial disease of the pilosebaceous follicles of the face and upper trunk, characterised by a variety of inflamed and non-inflamed lesions such as papules, pustules, nodules and open and closed comedones. It is associated with abnormal growth and metabolism of cutaneous propionibacteria such as P. acnes and P. granulosum. Currently known treatments include antibiotics—which bring the usual drawbacks of bacterial resistance and of unwanted side effects, particularly if administered systemically—and relatively harsh antibacterial agents such as benzoyl peroxide (known to induce skin irritation).

It has thus far proved difficult to identify antibacterial agents which are safe and suitable for use on the skin, and on other potentially sensitive epithelia, and yet are effective against problematic micro-organisms such as staphylococci (in particular antibiotic-resistant staphylococci) and propionibacteria.

It is therefore an aim of the present invention to provide antibacterial agents which can be used to overcome or at least mitigate such difficulties, and to provide alternative, often improved, antibacterial formulations for topical use.

### STATEMENTS OF THE INVENTION

According to a first aspect of the present invention there is provided the use of a benzoquinone or hydroquinone as a topical antibacterial agent.

The benzo/hydroquinone is preferably used as an agent against bacteria associated with (i.e., which cause, exacerbate or transmit) a skin or skin structure condition. It is preferably used against staphylococci and/or propionibacteria, more preferably against propionibacteria.

Certain quinones, for example alkyl-substituted hydroquinones such as 2-t-butyl hydroquinone (TBHQ), are already known for use as antioxidants. TBHQ itself has also been used as a stabilizer and preservative, for instance in foodstuffs, cosmetics and even adhesives. It has moreover been recognised as an antimycotic (DE-44 34 312). It has not to our knowledge however been used as an active agent against bacteria found on the skin or on any other external surface to which it is topically applied.

Thymoquinone, which occurs naturally as one of the constituents of black cumin seed extracts, is an alkyl-substituted benzoquinone which is known to possess both antibacterial (at least against S. aureus) and anti-inflammatory properties, and has also been shown to act as an anti-convulsant in a mouse model and as an anti-cancer agent. It is also known for use as a food preservative and generally as an antioxidant. See Kahsai A W, Master's thesis, East Tennessee State University, 2002; Saxena A P and Vyas K M in J. Econ. Taxon. Bot., 1986(8): 291-9; De M, Krishna D A and Banerjee A B in Phytoether Res., 1999(13): 616-8; Hosseinzadeh H and Parvardeh S, Phytomedicine 2004, 11(1): 56-64; and U.S. Pat. No. 6,218,434. Again to our knowledge, thymoquinone has not been used as an active agent against bacteria found on the skin, in particular against bacteria associated with skin and skin structure conditions.

Other quinones of various types have been disclosed for use as antimicrobial agents—see JP-2003-267910, JP-09255547 and U.S. Pat. No. 6,228,891—and as preservatives (JP-02202804). However again, to our knowledge, they have not been used against bacteria associated with skin and skin structure conditions.

According to the present invention, the benzo/hydroquinone is preferably used in a formulation which is suitable for topical application to, and/or contact with, the skin, in particular human skin. The benzo/hydroquinone is therefore preferably contained in a pharmaceutically acceptable vehicle which can safely be applied to, and/or contacted with, the skin and/or other epithelia. Ideally the formulation is suitable for topical application to, and/or contact with, areas such as the nares, eyes, scalp and vagina, as well as tissue areas within the ears and/or the oral cavity. Suitability for application to the skin, nares and tissue within the ears is most preferred, in particular the skin and nares.

A formulation which is “suitable for” topical application may also be adapted for topical application.

Suitable vehicles will be well known to those skilled in the art of preparing topical skin care or pharmaceutical preparations. The vehicle will typically be a fluid, which term includes a cream, paste, gel, lotion, ointment, foam or other viscous or semi-viscous fluid, as well as less viscous fluids such as might be used in sprays (for example for nasal use). The benzo/hydroquinone may be present in the form of a solution or suspension, the term “suspension” including emulsions and other multi-phase dispersions.

The benzo/hydroquinone may be carried in or on a delivery vehicle which is suitable for targeting or controlling its release at the intended site of administration. Such vehicles include liposomes and other encapsulating entities, for example niosomes, aspasomes, microsponges, microemulsions, hydrogels and solid lipid nanoparticles.

A benzoquinone is a cyclohexadiene-dione, containing two C═O groups in an unsaturated 6-membered ring. The four remaining carbon atoms may carry one or more substituents—in other words, the benzoquinone may be optionally substituted. The term “benzoquinone” is not however intended to embrace bi- or poly-cyclic quinones.

A hydroquinone (sometimes known as a hydroxyquinone) is a benzoquinone in which one or more—typically both—of the C═O groups is instead present as a C—OH group; in other words, it is typically a dihydroxy benzene, optionally substituted with one or more additional groups.

A benzoquinone may be present at least partly in the form of the corresponding hydroquinone, or vice versa, or either may exist at least partly as a radical in which one or more of the C═O or C—OH groups is present as C—O^{}. Such compounds may, depending in part on their local environment (for example pH), be present in the form of an equilibrium mixture of two or more such species, for instance of the benzoquinone and its corresponding hydroquinone. At alkaline pHs, for example, the compounds are more likely to be present in the form of the benzoquinones, whereas at acidic pHs they are more likely to be present as the hydroquinones. The presence of an oxidising agent may also induce at least partial conversion of a hydroquinone to the corresponding benzoquinone. The present invention thus embraces the use of a benzoquinone, a hydroquinone, a corresponding radical or any mixture of two or more such species.

The two C═O groups or C—OH groups of a benzo/hydroquinone may be positioned ortho, meta or para to one another. When positioned ortho to one another, this is known as a cyclohexadiene-1,2-dione or o-benzoquinone or, in the case of the corresponding hydroquinone, a catechol. When positioned meta to one another, this is known as a cyclohexadiene-1,3-dione or an m-benzoquinone or, in the case of the corresponding hydroquinone, a resorcinol. When positioned para to one another, this is known as a cyclohexadiene-1,4-dione or a p-benzoquinone or, in the case of the para-substituted HO-Ph-OH, simply as a p-hydroquinone.

Preferably the two C═O groups or C—OH groups are positioned ortho or para to one another, most preferably para.

The benzo/hydroquinone used in the invention may be, and in cases is preferably, substituted with one or more other groups such as those selected from alkyl, alkoxy, halogen, hydroxyl, nitro (—NO₂) and amine (—NR₂, where each R is independently either hydrogen or hydrocarbyl) groups. Such groups will be attached to carbon atoms in the cyclohexadiene ring of the quinone.

In general, an alkyl substituent may be either a straight or a branched chain alkyl group. It may be or contain cycloalkyl moieties. It may contain for instance from 1 to 12 carbon atoms, preferably from 1 to 10, more preferably from 1 to 8. An alkyl substituent is preferably a C₁ to C₆ alkyl group, more preferably a C₁ to C₅ or C₁ to C₄ alkyl group, yet more preferably selected from methyl, ethyl, iso-propyl and t-butyl. An alkoxy substituent is preferably a C₁ to C₆ alkoxy group, more preferably a C₁ to C₅ or C₁ to C₄ or C₁ to C₂ alkoxy group, most preferably methoxy. A halogen substituent may be selected from fluorine, chlorine and bromine, preferably fluorine or chlorine, most preferably chlorine. An amine substituent is preferably NH₂.

The benzo/hydroquinone is preferably substituted with at least one such substituent, which is preferably (at least in the case of a meta- or para-substituted benzo/hydroquinone) at the 2-position or (in the case of an ortho-substituted compound) at the 3-position. In some cases the benzo/hydroquinone may be substituted with two such substituents, in other cases with three or even four. It may be preferred for the benzo/hydroquinone to have either one or two such substituents, in some cases just one.

Particularly preferred are substituents selected from alkyl, alkoxy, halogen and nitro groups, or from alkyl, alkoxy and halogen groups, or from alkyl and halogen groups, or from alkyl and alkoxy groups. Most preferred substituents are the alkyl groups, in particular C₁ to C₄ alkyl groups.

The benzo/hydroquinone may be substituted with up to four alkyl groups, but in particular may be a mono- or di-alkyl benzo/hydroquinone.

The benzo/hydroquinone may for instance be substituted with one butyl group, which is preferably present at the 2-position; it may however be substituted with more than one butyl group, for instance two. A butyl group is preferably a t-butyl group.

The benzo/hydroquinone may be substituted with two butyl groups. These may for instance occupy the 2- and 5-positions, in particular where the benzo/hydroquinone is a para-benzo/hydroquinone. They may alternatively occupy the 3- and 5-positions, in particular where the benzo/hydroquinone is an ortho-benzo/hydroquinone. Again the butyl groups are preferably t-butyl groups.

Instead or in addition, the benzo/hydroquinone may be substituted with one methyl group, which is preferably present at the 2- or the 5-position; it may however be substituted with more than one methyl group, for instance two or three or even four. It may for instance be substituted with three methyl groups, which are preferably present at the 2-, 3- and 5-positions.

Instead or in addition, the benzo/hydroquinone may be substituted with one propyl group, which is preferably present at the 2-position. The benzo/hydroquinone may however be substituted with more than one propyl group, for instance two. A propyl group is preferably an iso-propyl group.

Instead or in addition, the benzo/hydroquinone may be substituted with one, two, three or even four ethyl groups, preferably one or two, more preferably one and yet more preferably at least one of them occupying the 2-position.

Although in many cases this will not be preferred, a hydroquinone may instead or in addition carry one or two (preferably one) substituents attached directly to the oxygen atom(s) of its C—OH groups (thus replacing the hydrogen atom(s) of hydroxyl group(s) on the cyclohexyl ring). For example, it may be substituted at one of the oxygen atoms with an alkyl group, preferred examples being as described above. The alkyl group may be hexyl, as in 1-o-hexyl-2,3,5-trimethyl hydroquinone (HTHQ).

The benzo/hydroquinone may in particular be selected from those listed in Examples 1 and 3 below. More particularly, it may be selected from TBHQ, thymoquinone and its derivatives such as dithymoquinone and thymohydroquinone, 2,5-di-t-butyl-p-hydroquinone, 3,5-di-t-butyl-o-benzoquinone, 2,3-dimethoxy-5-methyl-p-benzoquinone, 2,6-dimethoxy-p-benzoquinone, 2-methyl-p-hydroquinone, 2-methyl-p-benzoquinone, 2-chloro-p-hydroquinone, 2-bromo-p-hydroquinone, 2,5-dichloro-p-benzoquinone, 2,6-dichloro-p-benzoquinone, 2,3-difluoro-p-benzoquinone, 2-ethyl-p-hydroquinone, 2,3-dimethyl-p-hydroquinone, 2,5-dimethyl-p-benzoquinone, 2,6-dimethyl-p-hydroquinone, 2-t-butyl-p-benzoquinone, 2-chloro-5-methyl-p-benzoquinone and HTHQ.

It may for example be TBHQ or the corresponding 2-t-butyl-p-benzoquinone, or thymoquinone which is a para-benzoquinone substituted at the 2-position with an iso-propyl group and at the 5-position with a methyl group, or the corresponding thymohydroquinone.

In general it is preferred that the benzo/hydroquinone is not either an unsubstituted benzoquinone or an unsubstituted hydroquinone.

A benzo/hydroquinone used in the invention, in particular thymoquinone, dithymoquinone or thymohydroquinone, is ideally used in the form of the isolated quinone (whether naturally or synthetically derived, preferably the latter) rather than as part of a plant extract containing a number of different materials.

The benzo/hydroquinone may be of the type which is active as an antioxidant.

In cases it may be preferred for the quinone to be a hydroquinone, more preferably an alkyl-substituted hydroquinone. Of these, TBHQ is particularly preferred.

In one embodiment of the invention, the benzo/hydroquinone is used as an agent against staphylococci, in particular S. aureus. In this embodiment, the benzo/hydroquinone preferably:

a) is an ortho- or para-substituted benzo/hydroquinone, preferably the latter; and/or
b) is a hydroquinone, or at least a mixture of a hydroquinone and its corresponding benzoquinone which contains greater than 50%, more preferably greater than 60 or 70 or 80 or 90% w/w of the hydroquinone; and/or
c) has one or more, for example one or two, substituents selected from alkyl, alkoxy, halogen and nitro (preferred examples of such groups being as described above); and/or
d) has one or more, for example one or two, alkyl substituents, preferred examples being as described above; and/or
e) is substituted at least at the 2-position, more preferably with a substituent selected from alkyl, alkoxy, halogen and nitro (preferred examples of such groups being as described above), most preferably with an alkyl group; and/or
f) is not substituted with more than two electron-withdrawing groups such as halogen or nitro groups, and is more preferably not substituted with more than one such group; and/or
g) where it is a catechol or resorcinol, has two substituents, preferably alkyl substituents; and/or
h) is selected from the quinones listed in Example 1 below, more preferably from p-hydroquinone, p-benzoquinone, TBHQ, thymoquinone, thymohydroquinone, 2-bromo-p-hydroquinone, duroquinone (2,3,5,6-tetramethyl-p-benzoquinone), 2,5-di-t-butyl-p-hydroquinone, 3,5-di-t-butyl-o-benzoquinone, 2,3-dimethoxy-5-methyl-p-benzoquinone, 2,5-dimethyl-p-benzoquinone, 2,6-dimethoxy-p-benzoquinone, 2-t-butyl-p-benzoquinone, 2-methyl-p-hydroquinone, 2-methyl-p-benzoquinone, 2-chloro-p-benzoquinone, 2,5-dimethyl-resorcinol, 2,5-dichloro-p-hydroquinone, 3,5-di-t-butyl-catechol, 4-methyl-catechol, trimethyl-p-hydroquinone, 2-chloro-p-hydroquinone, 2-chloro-5-methyl-p-benzoquinone, 2,6-dichloro-p-hydroquinone, 2,3-dimethyl-p-hydroquinone, 2,6-dimethyl-p-hydroquinone, 2-ethyl-p-hydroquinone, 2,3-difluoro-p-hydroquinone, 2,5-dibromo-6-isopropyl-3-methyl-p-benzoquinone, 4,6-di-t-butyl-resorcinol and 1-O-hexyl-2,3,5-trimethyl-hydroquinone (HTHQ); and/or
i) is selected from TBHQ, thymoquinone, thymohydroquinone, 2,5-di-t-butyl-p-hydroquinone, 3,5-di-t-butyl-o-benzoquinone, 2,3-dimethoxy-5-methyl-p-benzoquinone, 2,5-dimethyl-p-benzoquinone, 2,6-dimethoxy-p-benzoquinone, 2-t-butyl-p-benzoquinone, 2-methyl-p-hydroquinone, 2-methyl-p-benzoquinone, 2,5-dimethyl-resorcinol, 2,5-dichloro-p-hydroquinone, 3,5-di-t-butyl-catechol, trimethyl-p-hydroquinone, 2-chloro-p-hydroquinone, 2-chloro-5-methyl-p-benzoquinone, 2,3-dimethyl-p-hydroquinone, 2,6-dimethyl-p-hydroquinone, 2-ethyl-p-hydroquinone, 2,3-difluoro-p-hydroquinone, 2,5-dibromo-6-isopropyl-3-methyl-p-benzoquinone, 4,6-di-t-butyl-resorcinol and HTHQ; and/or
j) is selected from TBHQ, thymoquinone, thymohydroquinone, 3,5-di-t-butyl-o-benzoquinone, 2,3-dimethoxy-5-methyl-p-benzoquinone, 2,5-dimethyl-p-benzoquinone, 2,6-dimethoxy-p-benzoquinone, 3,5-di-t-butyl-catechol, trimethyl-p-hydroquinone, 2-chloro-p-hydroquinone, 2,3-dimethyl-p-hydroquinone, 2,6-dimethyl-p-hydroquinone, 2-ethyl-p-hydroquinone, 2,5-dibromo-6-isopropyl-3-methyl-p-benzoquinone, 4,6-di-t-butyl-resorcinol and HTHQ; and/or
k) is selected from TBHQ, 3,5-di-t-butyl-o-benzoquinone, 2,3-dimethoxy-5-methyl-p-benzoquinone, 2,5-dimethyl-p-benzoquinone, 2,6-dimethoxy-p-benzoquinone, 3,5-di-t-butyl-catechol, trimethyl-p-hydroquinone, 2-chloro-p-hydroquinone, 2,3-dimethyl-p-hydroquinone, 2,6-dimethyl-p-hydroquinone, 2-ethyl-p-hydroquinone, 2,5-dibromo-6-isopropyl-3-methyl-p-benzoquinone, 4,6-di-t-butyl-resorcinol and HTHQ; and/or
l) is selected from 2-bromo-p-hydroquinone, 2,5-di-t-butyl-p-hydroquinone, 3,5-di-t-butyl-o-benzoquinone, 2,3-dimethoxy-5-methyl-p-benzoquinone, 2,6-dimethoxy-p-benzoquinone, 2-methyl-p-hydroquinone, 2,5-dichloro-p-hydroquinone, 2-chloro-p-hydroquinone, 2,6-dichloro-p-hydroquinone, 2-ethyl-p-hydroquinone, 2,3-difluoro-p-hydroquinone and p-hydroquinone, optionally together with 2,5-dibromo-6-isopropyl-3-methyl-p-benzoquinone; and/or
m) is selected from 2,5-di-t-butyl-p-hydroquinone, 3,5-di-t-butyl-o-benzoquinone, 2,3-dimethoxy-5-methyl-p-benzoquinone, 2,6-dimethoxy-p-benzoquinone, 2-methyl-p-hydroquinone, 2-chloro-p-hydroquinone and 2-ethyl-p-hydroquinone; and/or
n) is selected from 2,5-di-t-butyl-p-hydroquinone, 3,5-di-t-butyl-o-benzoquinone, 2-methyl-p-hydroquinone, 2-chloro-p-hydroquinone, 2,6-dimethyl-p-hydroquinone and 2-ethyl-p-hydroquinone, optionally together with TBHQ.

The above preferences may also apply more generally, for instance when the benzo/hydroquinone is used against micro-organisms other than staphylococci.

In another embodiment, the benzo/hydroquinone is used as an agent against propionibacteria, in particular P. acnes and more particularly for the treatment of acne, in which case it preferably:

a) is an ortho- or para-substituted benzo/hydroquinone, preferably the latter; and/or
b) is a benzoquinone, or at least a mixture of a benzoquinone and its corresponding hydroquinone which contains greater than 50%, more preferably greater than 60 or 70 or 80 or 90% w/w of the benzoquinone; and/or
c) has one or more, for example one or two, substituents selected from alkyl, alkoxy, halogen and nitro (preferred examples of such groups being as described above); and/or
d) has one or more, for example one or two, alkyl substituents, preferred examples being as described above; and/or
e) is substituted at least at the 2-position, more preferably with a substituent selected from alkyl, alkoxy, halogen and nitro (preferred examples of such groups being as described above), most preferably with an alkyl group; and/or
f) is not substituted with more than two electron-withdrawing groups such as halogen or nitro groups, and is more preferably not substituted with more than one such group; and/or
g) especially if it is a para-benzo/hydroquinone, is unsubstituted at the 5-position, or is substituted at the 5-position with a methyl group, more preferably the former; and/or
h) is selected from the quinones listed in Example 3 below, more preferably from p-benzoquinone, TBHQ, 3,5-di-t-butyl-o-benzoquinone, thymoquinone, thymohydroquinone, 2,3-dimethoxy-5-methyl-p-benzoquinone, 2,5-dimethyl-p-benzoquinone, 2-t-butyl-p-benzoquinone, 2,6-dimethoxy-p-benzoquinone, 2-methyl-p-benzoquinone, 2,5-dibromo-6-isopropyl-3-methyl-p-benzoquinone, 4,6-di-t-butyl-resorcinol, tetrachloro-p-hydroquinone, 3,5-di-t-butyl-catechol, HTHQ, 2,3-dimethyl-p-hydroquinone, 2-chloro-p-benzoquinone, 2-chloro-5-methyl-p-benzoquinone, 2,6-dimethyl-p-hydroquinone, 2,3-difluoro-p-hydroquinone, 2,5-dimethyl-resorcinol and 2-ethyl-p-hydroquinone; and/or
i) is selected from TBHQ, 3,5-di-t-butyl-o-benzoquinone, thymoquinone, 2,3-dimethoxy-5-methyl-p-benzoquinone, 2,5-dimethyl-p-benzoquinone, 2-t-butyl-p-benzoquinone, 2,6-dimethoxy-p-benzoquinone, 2-methyl-p-benzoquinone, 2,5-dibromo-6-isopropyl-3-methyl-p-benzoquinone, 4,6-di-t-butyl-resorcinol, 3,5-di-t-butyl-catechol, HTHQ, 2,3-dimethyl-p-hydroquinone, 2-chloro-p-benzoquinone, 2-chloro-5-methyl-p-benzoquinone, 2,6-dimethyl-p-hydroquinone, 2,3-difluoro-p-hydroquinone, 2,5-dimethyl-resorcinol and 2-ethyl-p-hydroquinone; and/or
j) is selected from TBHQ, 3,5-di-t-butyl-o-benzoquinone, thymoquinone, 2,3-dimethoxy-5-methyl-p-benzoquinone, 2,5-dimethyl-p-benzoquinone, 2-t-butyl-p-benzoquinone, 2-methyl-p-benzoquinone, 2,3-dimethyl-p-hydroquinone, 2-chloro-5-methyl-p-benzoquinone, 2,6-dimethyl-p-hydroquinone and 2-ethyl-p-hydroquinone, optionally together with 2,5-dibromo-6-isopropyl-3-methyl-p-benzoquinone; and/or
k) is selected from TBHQ, 3,5-di-t-butyl-o-benzoquinone, thymoquinone, 2,3-dimethoxy-5-methyl-p-benzoquinone, 2,5-dimethyl-p-benzoquinone, 2-t-butyl-p-benzoquinone, 2-methyl-p-benzoquinone and 2-chloro-5-methyl-p-benzoquinone.

The above preferences may also apply more generally, for instance when the benzo/hydroquinone is used against micro-organisms other than propionibacteria.

In all the cases described above, it may be preferred for the benzo/hydroquinone not to carry any substituents in addition to those specifically referred to in each case.

In some cases, in particular when it is used against staphylococci and more particularly when used against S. aureus, it may be preferred for the benzo/hydroquinone not to be thymoquinone, and in certain cases for it not to be either thymoquinone or thymohydroquinone.

In some cases, in particular when it is used against propionibacteria and/or to treat acne, and/or when it is to be applied to human skin, it may be preferred for the benzo/hydroquinone not to be unsubstituted p-hydroquinone.

In the context of the present invention, antibacterial activity encompasses activity against bacteria generally, whether Gram-positive or Gram-negative bacteria although preferably the former. It may be growth inhibitory activity or more preferably biocidal (i.e., lethal to the relevant organism). In particular it may be activity against bacteria associated with skin or skin-borne infections, more preferably against staphylococci (and in cases other Gram-positive cocci) and/or propionibacteria and/or suitably against other bacteria capable of causing, exacerbating or transmitting a skin or skin structure condition. Most preferably the formulation is active against strains of Staphylococcus aureus and/or Propionibacterium acnes.

In a particularly preferred embodiment of the invention, the benzo/hydroquinone is active against bacteria associated with acne, such as P. acnes and in some instances P. granulosum. It may instead or in addition be active against Gram-positive cocci, for example staphylococci (such as those listed in the examples below, in particular S. aureus) and streptococci (such as S. pyogenes) and in cases enterococci (such as E. faecalis and/or E. faecium, in particular the former).

In the context of the invention, activity against a particular species of micro-organism may be taken to mean activity against at least one, preferably two or more, strains of that species.

The benzo/hydroquinone is preferably active against bacteria, in particular staphylococci and/or propionibacteria, which are wholly or partially resistant to one or more antibiotics, for instance those which are in common clinical use. More particularly the benzo/hydroquinone is preferably active against one or more erythromycin-resistant, clindamycin resistant and/or tetracycline-resistant P. acnes strains of bacteria, and/or against one or more methicillin-resistant S. aureus (MRSA) strains, and/or against one or more vancomycin intermediate S. aureus (VISA) strains. It is preferably active at least against one or more erythromycin-resistant and/or tetracycline-resistant P. acnes strains.

Antibacterial activity may be measured in conventional manner, for instance using the tests described in the examples below. Generally tests for activity involve treating a culture of the relevant micro-organism with the candidate antibacterial compound, incubating the treated culture under conditions which would ordinarily support growth of the micro-organism, and assessing the level of growth, if any, which can occur in the presence of the candidate compound.

Preferably the benzo/hydroquinone used in the present invention has a minimum inhibitory concentration (MIC), at least against staphylococci and/or propionibacteria, of 150 μg/ml or less, more preferably 125 or 100 μg/ml or less, yet more preferably 70 or 50 or 40 or 30 or even 20 or 10 μg/ml or less, such as from 0.5 to 100 or 50 μg/ml. Its corresponding minimum biocidal concentration (MBC) is preferably 300 μg/ml or less, more preferably 150 μg/ml or less, yet more preferably 100 or 70 or 50 or 40 or 30 or even 20 or 10 μg/ml or less. Suitably the ratio of its MIC to its MBC is from 0.125 to 1, ideally from 0.5 to 1.

MIC and MBC values may be measured using conventional assay techniques, for instance as described in the examples below.

Preferably the benzo/hydroquinone retains antibacterial activity in the presence of at least one of, preferably two or more of, serum, lipid and salt (sodium chloride), for instance as tested in the examples below—these are species which can be present at the surface of the skin and hence performance in this context can be indicative of suitability for use in topical skin treatment formulations. Activity in the presence of lipid and sodium chloride can be especially important in the context of acne treatment; activity in the presence of serum and sodium chloride can be especially important in the context of the treatment or prevention of staphylococcal infections.

Ideally the benzo/hydroquinone retains at least some activity, preferably at least 50 or 60 or 70 or 80 or even 90% of its antibacterial activity, at least against staphylococci and/or propionibacteria, in the presence of at least one of, preferably two or more of, serum, lipid and salt. Yet more preferably the antibacterial activity of the benzo/hydroquinone, at least against staphylococci and/or propionibacteria, is potentiated by at least one of serum, lipid and sodium chloride. Most preferably the antibacterial activity of the benzo/hydroquinone is potentiated by lipid.

The concentration of the benzo/hydroquinone in an antibacterial formulation might suitably be 0.05% w/v or greater, preferably 0.1% w/v or greater, more preferably 0.5% w/v or greater. Its concentration might be up to 5% w/v, preferably up to 2.5% w/v, more preferably up to 2% w/v.

A formulation containing a benzo/hydroquinone, for use according to the invention, is preferably suitable for, and more preferably adapted for, topical administration to human or animal, in particular human, skin. It may also be suitable for, or adapted for, topical administration to other epithelia such as the nares, scalp, ears, eyes, vagina and oral cavity, in particular the nares and ears. It may take the form of a lotion, cream, ointment, foam, paste or gel or any other physical form known for topical administration, including for instance a formulation which is, or may be, applied to a carrier such as a sponge, swab, brush, tissue, skin patch, dressing or dental fibre to facilitate its topical administration. It may take the form of a nasal spray or of eye or ear drops. It may be intended for pharmaceutical (which includes veterinary) use, for example to treat skin infections or as a prophylactic against infections such as MRSA, and/or for cosmetic or other non-medical care purposes (for example, for general hygiene or cleansing).

The vehicle in which the benzo/hydroquinone is contained may be any vehicle or mixture of vehicles which is suitable for topical application; the type chosen will depend on the intended mode and site of application. Many such vehicles are known to those skilled in the art and are readily available commercially. Examples may for instance be found in Williams' “Transdermal and Topical Drug Delivery”, Pharmaceutical Press, 2003, and other similar reference books. See also Date, A. A. et al, Skin Pharmacol Physiol, 2006, 19(1): 2-16 for a review of topical drug delivery strategies for the treatment of acne.

As described above, the vehicle may be such as to target a desired site and/or time of delivery of the benzo/hydroquinone. It may for instance target the benzo/hydroquinone to the skin or hair follicles or to the anterior nares (the latter being particularly suitable when the benzo/hydroquinone is used as a preventative treatment against MRSA or other staphylococci). It may delay or otherwise control release of the benzo/hydroquinone over a particular time period. The benzo/hydroquinone may be microencapsulated, for instance in liposomes—particularly suitable liposomes, for topical use, are those made from stratum corneum lipids, e.g., ceramides, fatty acids or cholesterol.

In some cases a polar vehicle may be preferred. Where the benzo/hydroquinone-containing formulation is intended for use on the skin, in particular to treat skin and skin structure infections, the vehicle may be primarily non-aqueous, although in the case of an anti-acne treatment an aqueous vehicle may be used. The vehicle may be surface-active, in particular when it is intended for use in treating surfaces, for instance to cleanse instruments or working areas in particular against staphylococci. In cases the vehicle may be alcohol-based or silicon-based.

The formulation may contain standard excipients and other additives known for use in pharmaceutical or veterinary formulations, in particular topical skin care formulations. Examples include emollients, perfumes, antioxidants, preservatives and stabilisers; others may be found in Williams' “Transdermal and Topical Drug Delivery”, supra.

It may further contain additional active agents such as antimicrobial agents. Where the formulation is intended for topical application to the skin, in particular to treat skin and skin structure infections and/or to treat conditions such as acne or infected atopic eczema, it may additionally contain one or more agents selected from anti-acne agents, keratolytics, comedolytics, anti-inflammatories, anti-proliferatives, antibiotics, anti-androgens, sebostatic agents, anti-pruritics, immunomodulators, agents which promote wound healing and mixtures thereof, it may instead or in addition contain one or more agents selected from sunscreens, moisturisers, emollients and mixtures thereof. Generally speaking a formulation for use according to the invention may contain one or more agents which enhance the activity of another active agent present in the formulation, or reduce a side effect of such an active, or improve patient compliance on administration of the formulation.

An additional antimicrobial agent may for example be selected from the group consisting of biocides, disinfectants, antiseptics, antibiotics, antimicrobially active antioxidants and mixtures thereof, it is preferably active as a bactericide, in particular against propionibacteria and/or staphylococci. It may be active as an anti-mycotic. It may be a peroxide, in particular a diacyl peroxide such as benzoyl peroxide.

It may however be preferred for the benzo/hydroquinone to be the only active agent in the formulation, or at least to be the only antibacterially active agent.

When used according to the invention, the benzo/hydroquinone may be in a formulation which is suitable for, more preferably adapted for, use on a surface other than living tissue, for instance to treat floors or walls (whether internal or external), work surfaces or instruments, to disinfect contact lenses or to cleanse hair or teeth or nails so as to reduce microbe levels. It may be suitable for application to growing or harvested crops, foodstuffs, non-living tissue (for instance for use as a preservative), bedding or clothing (for instance for bio-agent decontamination). In these cases the excipients, vehicles and/or other additives included with the benzo/hydroquinone may be different to those included in a topical skin care formulation, but again may be conventional as known for use in such contexts.

The benzo/hydroquinone-containing formulation may be incorporated into, and hence applied in the form of, another product such as a cosmetic, a skin or hair care preparation, a pharmaceutical (which includes veterinary) preparation, a toiletry product (for instance a bath or shower additive or a cleansing preparation), a laundry or other fabric treatment product or an agricultural or horticultural product.

According to a second aspect of the present invention there is provided a benzoquinone or hydroquinone, or a formulation containing a benzo/hydroquinone, for use in the topical treatment of a condition which is either caused, exacerbated or transmitted by bacterial activity, in particular staphylococcal and/or propionibacterial activity. The condition is preferably a skin or skin structure condition.

In the context of the present invention, treatment of a condition encompasses both therapeutic and prophylactic treatment, of either an infectious or a non-infectious condition, in either a human or an animal and in particular on the skin. It thus involves use of the benzo/hydroquinone as a bactericide, most particularly against staphylococci and/or propionibacteria.

Skin and skin structure conditions which might be treated according to the invention include acne, infected atopic eczema, superficial infected traumatic lesions, wounds, burns, ulcers, folliculitis, mycoses and other superficial primary and secondary skin and skin structure infections. In particular the benzo/hydroquinone may be for use in treating acne or acne lesions (for instance, to reduce ache-related scarring).

Treatment of acne encompasses the treatment and/or prevention of lesions and/or scarring associated with acne. Acne is a multifactorial disease of the pilosebaceous follicles of the face and upper trunk, characterised by a variety of inflamed and non-inflamed lesions such as papules, pustules, nodules and open and closed comedones. Its treatment can therefore encompass the treatment of any of these symptoms.

In general, a benzo/hydroquinone will be used, according to the invention, for the treatment of symptoms which are directly due to acne rather than for instance infections which may arise as a consequence of treating acne with other actives such as antibiotics.

In the context of the present invention, “skin or skin structure condition” may in some cases encompass a condition affecting other epithelia such as in the nares, scalp, vagina, eyes, ears or oral cavity. In most cases, however, a skin or skin structure condition will be one affecting the skin or skin structure directly.

The benzo/hydroquinone may also be for use as a therapeutic or prophylactic treatment for any area of the body—in particular the skin or nares—against staphylococci, which might otherwise cause for example MRSA-associated infections, or infections in pre-existing lesions such as eczematous lesions.

Other examples of conditions which may be treated in accordance with the fifth aspect of the invention include oral, ocular, aural, nasal and vaginal conditions. Again, treatment of such conditions encompasses both therapeutic and prophylactic treatment, of either an infectious or a non-infectious condition, in either a human or an animal but in particular a human. In particular it encompasses the prophylactic treatment of any area of the body, in particular the skin or nares, against microbial and especially bacterial infections.

Treatment of a condition may involve complete or partial eradication of the condition, removal or amelioration of associated symptoms, arresting subsequent development of the condition, and/or prevention of, or reduction of risk of, subsequent occurrence of the condition.

According to a third aspect, the present invention provides the use of a benzoquinone or hydroquinone in the manufacture of a medicament for the topical treatment of a condition, in particular a skin or skin structure condition, which is either caused by, exacerbated by or transmitted by bacterial activity, in particular staphylococcal and/or propionibacterial activity. Such a condition may be for example an MRSA infection, or acne.

The invention further provides, according to a fourth aspect, the use of a benzoquinone or hydroquinone in the manufacture of an antibacterial formulation for topical use.

A fifth aspect provides a method for controlling the growth of a bacterial micro-organism, in particular a staphylococcal bacterium or Propionibacterium, the method comprising topically applying, to an area infected or suspected to be infected or capable of becoming infected with the micro-organism, a benzoquinone or hydroquinone.

In this context, “controlling the growth” of a micro-organism embraces inhibiting or preventing its growth, whether completely or partially, as well as killing either completely or partially a culture of the organism. It also embraces reducing the risk of subsequent growth of the organism in the area being treated. The method of the invention may thus be used to treat an existing occurrence of the organism or to prevent a potential subsequent occurrence.

Again the area to which the benzo/hydroquinone is applied will typically be a surface such as human or animal tissue, in particular the skin or nares, typically of a living human or animal. In this case the benzo/hydroquinone may be applied for therapeutic purposes or for non-therapeutic (e.g., purely cosmetic) purposes. Alternatively it may be a non-living surface such as in a hospital or food preparation area. For example the method of the fifth aspect of the invention may be used to treat work surfaces, surgical or other instruments, surgical implants or prostheses, contact lenses, foods, crops, industrial plant, floors and walls (both internal and external), bedding, furniture, clothing and many other surfaces.

A sixth aspect of the invention provides a method for the treatment, in a human or animal—typically human—patient, of a condition which is either caused, exacerbated or transmitted by bacterial (preferably staphylococcal and/or propionibacterial) activity, the method involving the topical application of a benzoquinone or hydroquinone. Again the condition is suitably a skin or skin structure condition, preferably acne. The condition may be a staphylococcal infection. The benzo/hydroquinone may be applied to the skin, or in cases to other epithelia such as in particular the nares.

The method of the sixth aspect of the invention embraces a method for controlling bacterial growth in or on a human or animal patient, the bacteria typically being staphylococci and/or propionibacteria and the growth typically being controlled on the skin or in cases on other epithelia such as the nares.

The treatment or control may be carried out for prophylactic purposes.

Preferred features of the second and subsequent aspects of the invention may be as described in connection with any of the other aspects.

Other features of the present invention will become apparent from the following examples. Generally speaking the invention extends to any novel one, or any novel combination, of the features disclosed in this specification (including any accompanying claims and drawings). Moreover unless stated otherwise, any feature disclosed herein may be replaced by an alternative feature serving the same or a similar purpose.

Embodiments of the present invention will now be illustrated by means of the following experimental examples.

### DETAILED DESCRIPTION

Experimental tests were conducted to determine the antimicrobial activities of benzoquinone- or hydroquinone-containing formulations according to the invention.

Test Micro-Organisms

The following test micro-organisms were used.

1. Staphylococcus aureus—the principal staphylococcal test micro-organism used in these studies was Staphylococcus aureus ATCC 29213. This strain is the one recommended for QC/QA purposes in Minimum Inhibitory Concentration (MIC) assays by the US Clinical and Laboratory Standards Institute (formerly the NCCLS), an FDA-recognised body. S. aureus ATCC 29213 is susceptible to beta-lactam antibiotics such as methicillin and to many other antibiotics in clinical use worldwide today.

Other staphylococcal strains were also tested, as described in Example 2 below. These included certain antibiotic resistant staphylococci, such as the methicillin resistant S. aureus (MRSA) strains EMRSA-15 and EMRSA-16, both available from the Central Public Health Laboratory (CPHL), Colindale, UK. These strains are resistant not only to all beta-lactams but also to a number of other antibiotics in clinical use, making them a serious threat to human health. They are also responsible for the majority (>95%) of hospital-acquired MRSA infections in the UK.

S. aureus and other staphylococci are common causes of a wide range of skin, skin structure and wound infections. S. aureus itself is also known to exacerbate eczema.

2. Propionibacterium spp.—the principal propionibacterial strain used in these studies was Propionibacterium acnes NCTC 737. This is the type strain of the genus; it is fully susceptible to antibiotics.

The propionibacteria are clinically significant due to their involvement in acne. This is a very common, complex and multi-factorial skin disease in which P. acnes and other Propionibacterium spp. (for example P. granulosum) play key roles. They are also opportunistic pathogens in compromised hosts.

Other propionibacterial strains were also tested, as described in Example 4 below. These included certain antibiotic resistant propionibacteria, such as the two P. acnes strains designated PRP-010 and PRP-039 which are resistant respectively to macrolides-lincosamides-streptogramins-ketolides (MLSK) and to macrolides-lincosamides-streptogramins (MLS) and tetracycline—in other words, PRP-010 is resistant to erythromycin and clindamycin, and PRP-039 to erythromycin, clindamycin and tetracycline.

In addition, certain strains of P. granulosm, another bacterium involved in acne, were also tested in Example 4.

3. Streptococcus pyogenes ATCC 12344—this is a Gram-positive facultatively aerobic bacterium; it is a member of the group A β-haemolytic streptococci which are occasional upper respiratory tract commensals in humans (10% of children, commensal carriage uncommon in adults, its main occurrence is as the agent of tonsillitis). It is clinically significant in the context of skin infections, being a causative agent of cellulitis, necrotising fasciitis, impetigo, erysipelas, wound infections and scarlet fever. Penicillin and erythromycin are the standard treatments for infections involving S. pyogenes. However certain conditions, most notably necrotising fasciitis, may not respond to the standard antibiotic treatments. S. pyogenes is also a major cause of upper respiratory tract infections.

4. Enterococcus faecalis ATCC 29212—this is another Gram-positive bacterium belonging to the genus Enterococcus. Enterococci have similar properties to streptococci, but differ in their ability to grow on bile-salt containing media such as MacConkey's Agar. Their principal habitat is the mammalian gastrointestinal tract. They cause a number of important infections including endocarditis, urinary tract infections and abscesses. In the context of skin, they are frequently isolated from wound infections. Unlike the streptococci, the enterococci have developed widespread resistance to penicillin. More recently, E. faecalis and E. faecium strains have also developed resistance to the glycopeptide antibiotics such as vancomycin. Vancomycin-Resistant Enterococci (VRE), principally vanA strains of E. faecium, now represent a serious healthcare-acquired infection hazard in the USA, Japan and Western Europe.

5. Haemophilus influenzae ATCC 49247—a member of the genus Haemophilus, these organisms are Gram-negative bacilli which grow under aerobic or anaerobic conditions but require media with special supplements for laboratory culture. Strains of H. influenzae may be capsulated or non-capsulated. The capsulate strains are divided into serotypes, (designated b,c,d,e,f), on the basis of capsular antigens. The serotype H. influenzae type b is an important cause of invasive infections in humans, particularly in young children. The non-capsulate strains are common commensals in the upper respiratory tract, although there are also some healthy carriers of the type b strain. In the context of skin and topical treatments, H. influenzae can be a cause of cellulitis and otitis media (middle ear infection), again particularly in young children.

Most H. influenzae strains produce β-lactamase and are resistant to ampicillin. Until recently, chloramphenicol was a common treatment. However, in some countries, resistance is increasing and the cephalosporin antibiotic cefotaxime is now used as a first-line treatment.

Activity observed against these micro-organisms is expected to be a reasonable qualitative predictor of antimicrobial activity generally, but in particular of activity against micro-organisms responsible for skin and skin structure infections, including acne.

The staphylococci and enterococci were cultured and maintained on Mueller-Hinton Medium (agar and broth) at pH 7.2; they were incubated aerobically at 37° C. for 24 hours.

The Propionibacterium spp organisms were cultured and maintained on Wilkins-Chalgren Anaerobe Medium (agar and broth) at pH 6.0; cultures were incubated anaerobically at 37° C. for 72 hours.

The S. pyogenes strain was cultured and maintained on Mueller-Hinton agar with 5% v/v defibrinated horse blood, and grown in Mueller-Hinton broth supplemented with 5% lysed horse blood (lysed horse blood was prepared as described in the NCCLS M7-A6 Vol. 23 No. 2); cultures were incubated aerobically at 37° C. for 24 hours.

The H. influenzae strain was cultured and maintained on Haemophilus Test Medium (HTM) agar/broth (the composition of which is as described in NCCLS M7-A6 Vol. 23 No. 2); cultures were incubated in a CO₂ atmosphere at 37° C. for 24 hours.

The following tests were carried out to assess antimicrobial activity against these organisms.

(a) Minimum Inhibitory Concentration (MIC) Assay

This is a standard international method for quantitatively assessing the antimicrobial activity of a compound in a liquid medium. The method used a 96-well microtitre plate, capable of holding about 200 μl of liquid per well. The wells contained liquid culture medium and ranges of decreasing concentrations of the relevant test compound in doubling dilutions (eg, 1000, 500, 250, 125 . . . μg/ml, etc. down to 1.95 μg/ml). The culture media were as described above for the relevant test organisms.

The wells were inoculated with a liquid suspension of freshly grown micro-organism and incubated under the conditions described above. After incubation, the microtitre plate was examined visually (with the aid of a light box) for cloudiness in each well, which would indicate microbial growth. The MIC value was recorded as the lowest concentration of test compound required to inhibit microbial growth, i.e., the lowest concentration for which the liquid in the well remained clear.

The assays were conducted in duplicate and included both negative (culture medium only) and positive (culture medium, diluting solvent and inoculum) controls.

Since inhibition does not necessarily indicate killing of microbial cells, merely that growth as visible to the naked eye has been inhibited, it is desirable to conduct a further test (the MBC assay described below) to establish the concentration of the test compound needed to kill the test organism.

(b) Minimum Bactericidal Concentration (MBC) Assay

This assay, normally carried out after an MIC assay, determines the minimum concentration of a compound that is lethal to the micro-organism being tested.

Following an MIC assay, a 5 μl sample was withdrawn from the first microtitre well that showed positive growth and from all the subsequent wells that showed no growth. These samples were then individually sub-cultured on non-selective agar medium, under the incubation conditions described above. Following incubation they were examined visually for bacterial growth. The MBC was taken to be the lowest test compound concentration for which the incubated sample showed no growth.

The ratio of MIC to MBC should ideally be as close to 1 as possible. This facilitates selection of the lowest possible effective concentration of a test compound with a reduced risk of selecting a sub-lethal concentration which could promote resistance or be overcome by natural (i.e., innate) antimicrobial resistance.

(c) Disc Diffusion Assay (DDA)

This is an internationally recognised standard method for qualitatively assessing the antimicrobial activity of a compound.

A sterile paper disc was impregnated with a sample of the test compound and a minimum of 30 minutes allowed for the solvents to evaporate as far as possible. The disc was then placed on an agar plate onto which the test micro-organism had been inoculated. The plate was then incubated under the conditions described above, following which it was examined visually for signs of bacterial growth. If the test compound had antimicrobial activity, a circular zone of no growth would be obtained around the disc. The diameter of this zone of “inhibition” was measured using a ProtoCOL™ automated zone sizer (Synbiosis, Cambridge, UK). In general, a greater diameter and/or area of the zone of inhibition indicates a greater antimicrobial activity in the relevant test compound, although other factors such as test compound mobility through the agar gel may also influence the result.

The area of the zone of inhibition was calculated from the measured zone diameter (D) using the formula π(D/2)².

(d) Supplemented Disc Diffusion Assays

DDA tests may be carried out using an agar gel supplemented with blood, lipid and/or salt to simulate some of the major components present in human skin and to assess whether these substances might reduce the antimicrobial activity observed for the test compounds. Performance under these conditions can provide a more reliable indication of activity on topical application. For assays conducted using S. aureus strains, the supplements were defibrinated horse blood (5% v/v), lipid (Tween™ 80 at 1% v/v) and sodium chloride (100 mM). For those using Propionibacterium spp. strains, the supplements were lipid (Tween™ 80 at 1% v/v) and sodium chloride (100 mM).

### EXAMPLE 1

### Activity Against S. aureus ATCC 29213

The following experiments all used S. aureus ATCC 29213 as the test organism. MIC, MBC and DDA assays, as described above, were carried out using a range of different benzoquinones and hydroquinones. Supplemented DDA assays, in the presence of salt, lipid and blood, were also conducted.

For most DDA assays, 200 μg of each compound was loaded onto each disc. The exceptions were the assays for thymoquinone, in which only 50 μg of the benzoquinone was used, and those for 2,6-dimethoxy-p-benzoquinone, in which 100 μg of the relevant benzoquinone was used. The solvents used were ethanol (for TBHQ, thymoquinone, duroquinone, p-hydroquinone, p-benzoquinone and thymohydroquinone) and DMSO for all other test compounds.

The MIC and MBC results are shown in Table 1 below, the DDA results in Table 2. All results are collated from a number of experiments.

TABLE 1

| | MIC | MBC | MIC/MBC |
|---|---|---|---|
| Test compound | (μg/ml) | (μg/ml) | ratio |
| TBHQ | 7.8 | 7.8 | 1 |
| 2,5-dibromo-6-isopropyl-3-methyl-p- | 0.98 | 1.95 | 0.5 |
| benzoquinone | | | |
| 2,6-dimethyl-p-hydroquinone | 3.9 | 7.8 | 0.5 |
| 1-o-hexyl-2,3,5-trimethyl-p-hydroquinone | 3.9 | 7.8 | 0.5 |
| thymoquinone | 7.8 | 15.6 | 0.5 |
| 3,5-di-t-butyl-o-benzoquinone | 7.8 | 7.8 | 1 |
| 2,3-dimethoxy-5-methyl-p-benzoquinone | 7.8 | 15.6 | 0.5 |
| 2,5-dimethyl-p-benzoquinone | 7.8 | 15.6 | 0.5 |
| 2,6-dimethoxy-p-benzoquinone | 7.8 | 15.6 | 0.5 |
| 3,5-di-t-butylcatechol | 7.8 | 7.8 | 1 |
| 2,5,6-trimethyl-p-hydroquinone | 7.8 | 31.25 | 0.25 |
| 2-chloro-p-hydroquinone | 7.8 | 7.8 | 1 |
| 2,3-dimethyl-p-hydroquinone | 7.8 | 7.8 | 1 |
| 2-ethyl-p-hydroquinone | 7.8 | 15.6 | 0.5 |
| 4,6-di-t-butylresorcinol | 7.8 | 15.6 | 0.5 |
| thymohydroquinone | 7.8 | 15.6 | 0.5 |
| 2,5-di-t-butyl-p-hydroquinone | 15.6 | 15.6 | 1 |
| 2-t-butyl-p-benzoquinone | 15.6 | 15.6 | 1 |
| 2-methyl-p-hydroquinone | 15.6 | 15.6 | 1 |
| 2-methyl-p-benzoquinone | 15.6 | 15.6 | 1 |
| 2,5-dimethylresorcinol | 15.6 | >250 | <0.06 |
| 2,5-dichloro-p-hydroquinone | 15.6 | 15.6 | 1 |
| 2-chloro-5-methyl-p-benzoquinone | 15.6 | 15.6 | 1 |
| 2,3-difluoro-p-hydroquinone | 15.6 | 15.6 | 1 |
| 2-bromo-p-hydroquinone | 31.25 | 31.25 | 1 |
| duroquinone (tetramethyl-p-benzoquinone) | 31.25 | 62.5 | 0.5 |
| p-benzoquinone | 31.25 | 31.25 | 1 |
| 2-chloro-p-benzoquinone | 31.25 | 31.25 | 1 |
| 4-methylcatechol | 31.25 | 31.25 | 1 |
| 2,6-dichloro-p-hydroquinone | 31.25 | 31.25 | 1 |
| p-hydroquinone | 62.5 | 62.5 | 1 |
| 2,6-di-t-butyl-p-benzoquinone | 62.5 | 250 | 0.25 |
| 4-t-butylcatechol | 62.5 | 62.5 | 1 |
| 3-methylcatechol | 62.5 | 62.5 | 1 |
| tetrafluoro-p-hydroquinone | 125 | 125 | 1 |
| tetrachloro-p-hydroquinone | 125 | 125 | 1 |
| 4-nitrocatechol | 125 | >250 | <0.5 |
| 2-nitroresorcinol | 125 | >250 | <0.5 |

TABLE 2

| | | DDA + | DDA + | DDA + |
|---|---|---|---|---|
| | DDA | salt | lipid | blood |
| Test compound | (mm) | (mm) | (mm) | (mm) |
| TBHQ | 41.77 | 54.16 | 31.44 | 10.89 |
| | (±2.01) | | | |
| 2,5-dibromo-6-isopropyl-3-methyl-p-benzoquinone | 39.53 | 42.33 | 23.04 | 19.30 |
| | (±0.31) | | | |
| 2,6-dimethyl-p-hydroquinone | 40.88 | 43.89 | 47.94 | 25.53 |
| | (±1.40) | | | |
| 1-o-hexyl-2,3,5-trimethyl-p-hydroquinone | 8.61 | 8.72 | 0.0 | 0.0 |
| | (±0.18) | | | |
| thymoquinone | 15.64 | 20.54 | 18.99 | 0.0 |
| | (±1.03) | | | |
| 3,5-di-t-butyl-o-benzoquinone | 23.45 | 23.04 | 17.43 | 15.88 |
| | (±1.26) | | | |
| 2,3-dimethoxy-5-methyl-p-benzoquinone | 26.77 | 25.84 | 26.46 | 19.92 |
| | (±0.62) | | | |
| 2,5-dimethyl-p-benzoquinone | 68.84 | 73.10 | 75.29 | 26.24 |
| | (±0.95) | | | |
| 2,6-dimethoxy-p-benzoquinone | 22.18 | 22.18 | 23.74 | 17.18 |
| | (±0.31) | | | |
| 3,5-di-t-butylcatechol | 18.75 | 19.69 | 10.94 | 11.88 |
| | (±0.63) | | | |
| 2,5,6-trimethyl-p-hydroquinone | 63.40 | 61.63 | 56.97 | 0.0 |
| | (±1.47) | | | |
| 2-chloro-p-hydroquinone | 17.54 | 19.30 | 16.81 | 13.70 |
| | (±0.36) | | | |
| 2,3-dimethyl-phydroquinone | 33.41 | 34.24 | 35.80 | 14.32 |
| | (±0.48) | | | |
| 2-ethyl-p-hydroquinone | 21.06 | 24.59 | 23.04 | 17.74 |
| | (±0.48) | | | |
| 4,6-di-t-butylresorcinol | 8.09 | 8.40 | 0.0 | 0.0 |
| | (±0.31) | | | |
| thymohydroquinone | 57.62 | 57.59 | 41.71 | 10.27 |
| | (±2.67) | | | |
| 2,5-di-t-butyl-p-hydroquinone | 10.06 | 11.21 | 10.58 | 10.89 |
| | (±0.48) | | | |
| 2-t-butyl-p-benzoquinone | 58.53 | 54.67 | >90 | 10.93 |
| | (±0.79) | | | |
| 2-methyl-p-hydroquinone | 25.70 | 22.49 | 24.68 | 18.74 |
| | (±0.64) | | | |
| 2-methyl-p-benzoquinone | 35.09 | 34.99 | 35.62 | 15.93 |
| | (±0.18) | | | |
| 2,5-dimethylresorcinol | 16.67 | 16.88 | 13.75 | 0.0 |
| | (±0.72) | | | |
| 2,5-dichloro-p-hydroquinone | 19.90 | 18.75 | 15.63 | 13.44 |
| | (±0.18) | | | |
| 2-chloro-5-methyl-p-benzoquinone | 34.55 | 34.24 | 33.93 | 17.74 |
| | (±1.32) | | | |
| 2,3-difluoro-p-hydroquinone | 15.77 | 15.56 | 14.63 | 10.27 |
| | (±0.48) | | | |
| 2-bromo-p-hydroquinone | 20.75 | 18.99 | 17.43 | 14.94 |
| | (±0.36) | | | |
| duroquinone | 13.28 | 16.50 | 16.19 | 0.0 |
| | (±0.18) | | | |
| p-benzoquinone | 29.34 | 27.49 | 29.05 | 20.31 |
| | (±0.19) | | | |
| 2-chloro-p-benzoquinone | 25.31 | 26.25 | 22.81 | 17.19 |
| | (±0.54) | | | |
| 4-methylcatechol | 18.33 | 20.94 | 20.00 | 0.0 |
| | (±0.18) | | | |
| 2,6-dichloro-p-hydroquinone | 16.29 | 16.19 | 14.32 | 11.83 |
| | (±0.48) | | | |
| p-hydroquinone | 18.94 | 16.61 | 15.56 | 14.01 |
| | (±0.84) | | | |
| 2,6-di-t-butyl-p-benzoquinone | 0.0 | 0.0 | 9.37 | 0.0 |
| | (±0.0) | | | |
| 4-t-butylcatechol | 12.04 | 13.07 | 10.89 | 9.65 |
| | (±0.18) | | | |
| 3-methylcatechol | 19.82 | 20.23 | 18.68 | 0.0 |
| | (±0.36) | | | |
| tetrafluoro-p-hydroquinone | 10.83 | 11.25 | 9.38 | 0.0 |
| | (±0.18) | | | |
| tetrachloro-p-hydroquinone | 16.25 | 16.88 | 14.69 | 9.38 |
| | (±0.0) | | | |
| 4-nitrocatechol | 18.88 | 18.99 | 16.50 | 11.83 |
| | (±1.18) | | | |
| 2-nitroresorcinol | 10.48 | 12.76 | 0.0 | 0.0 |
| | (±0.48) | | | |

The data in Tables 1 and 2 show that each of the quinones is active against S. aureus ATCC 29213, some strongly so (in particular TBHQ, 2,5-dibromo-6-isopropyl-3-methyl-p-benzoquinone, 2,6-dimethyl-p-hydroquinone, thymoquinone, 3,5-di-t-butyl-o-benzoquinone, 2,3-dimethoxy-5-methyl-p-benzoquinone, 2,5-dimethyl-p-benzoquinone, 2,6-dimethoxy-p-benzoquinone, 2,5,6-trimethyl-p-hydroquinone, 2,3-dimethyl-p-hydroquinone, 2-ethyl-p-hydroquinone, thymohydroquinone, 2-t-butyl-p-benzoquinone, 2,5-dimethyl-p-benzoquinone and 2,5,6-trimethyl-p-hydroquinone—these have a MIC of ≦7.8 μg/ml or a zone diameter of ≧50 mm on unsupplemented Mueller-Hinton agar).

Those likely to be of most interest for use against staphylococcal infections of the skin are the benzo/hydroquinones whose activity is least strongly affected by the presence of serum and salt (for example, those showing a reduction in zone size of <50% for both)—these include for example 2,6-dimethyl-p-hydroquinone, 3,5-di-t-butyl-o-benzoquinone, 2,3-dimethoxy-5-methyl-p-benzoquinone, 2,6-dimethoxy-p-benzoquinone, 2-ethyl-p-hydroquinone, 3,5-di-t-butyl-catechol and 2-chloro-p-hydroquinone.

### EXAMPLE 2

### Activity Against Other Staphylococci

The activity of the alkyl-substituted hydroquinone TBHQ was tested against other staphylococcal bacterial strains, including some with known antibiotic resistance. MIC, MBC and DDA assays were carried out as described above for each of the strains.

For the DDA assays, 200 μg of TBHQ was loaded onto each disc. The solvent used for the TBHQ was ethanol.

The results are shown in Table 3 below; all are collated from a number of experiments. The table indicates the resistance phenotype for each of the test strains, some of which are resistant to many commonly used antibiotics.

TABLE 3

| | Resistance | MIC | MBC | DDA |
|---|---|---|---|---|
| Test organism | phenotype | (μg/ml) | (μg/ml) | (mm) |
| Staphylococcus | ND | 7.8 | 31.25 | 53.07 |
| simulans ATCC 27848 | | | | (±0.83) |
| Staphylococcus | ND | 7.8 | 15.62 | 49.72 |
| xylosus ATCC 29971 | | | | (±0.48) |
| Staphylococcus cohnii | ND | 3.9 | 7.8 | 60.40 |
| ATCC 29974 | | | | (±3.95) |
| Staphylococcus | ND | 3.9 | 7.8 | 51.08 |
| haemolyticus ATCC | | | | (±1.73) |
| 29970 | | | | |
| Staphylococcus | ND | 3.9 | 7.8 | 54.43 |
| warneri ATCC 27836 | | | | (±4.10) |
| Staphylococcus capitis | ND | 1.95-3.9 | 3.9 | 73.90 |
| ATCC 27840 | | | | (±2.67) |
| Staphylococcus. | ND | 1.95 | 3.9 | 64.59 |
| hominis ATCC 27844 | | | | (±1.42) |
| Staphylococcus | ND | 0.98 | 1.95 | 80.18 |
| auricularis ATCC | | | | (±0.48) |
| 33753 | | | | |
| Staphylococcus aureus | ND | 3.9 | 7.8 | 49.24 |
| ATCC 12600 | | | | (±3.52) |
| S. aureus ATCC | ND | 3.9 | 7.8 | 51.24 |
| 12600-U | | | | (±2.15) |
| S. aureus ATCC 12601 | ND | 7.8 | 15.6 | 57.34 |
| | | | | (±0.79) |
| S. aureus ATCC 12602 | ND | 3.9 | 7.8 | 57.66 |
| | | | | (±3.82) |
| S. aureus ATCC 12604 | ND | 7.8 | 7.8 | 55.03 |
| | | | | (±2.68) |
| S. aureus ATCC 12605 | ND | 3.9 | 3.9 | 59.55 |
| | | | | (±1.42) |
| S. aureus ATCC 12606 | ND | 7.8 | 7.8 | 53.34 |
| | | | | (±1.67) |
| S. aureus ATCC 12607 | ND | 3.9 | 3.9 | 54.50 |
| | | | | (±1.82) |
| S. aureus ATCC 29213 | ND | 7.8 | 15.6 | 46.79 |
| | | | | (±1.30) |
| S. aureus ATCC 25923 | ND | 7.8 | 7.8 | 44.70 |
| | | | | (±0.90) |
| S. aureus CPHL | Met/β-lactams* | 3.9 | 3.9 | 55.14 |
| EMRSA 15 | | | | (±1.49) |
| S. aureus CPHL | Met/β-lactams* | 3.9 | 3.9 | 67.72 |
| EMRSA 16 | | | | (±3.10) |
| S. aureus CPHL | Met/β-lactams* | 1.95 | 3.9 | 51.05 |
| EMRSA 17 | | | | (±0.48) |
| S. aureus CPHL VISA | Van* | 3.9 | 7.8 | 50.73 |
| Mu3 | (intermediate) | | | (±1.49) |
| S. aureus CPHL VISA | Van* | 3.9 | 7.8 | 50.11 |
| Mu50 | (intermediate) | | | (±1.55) |
| S. aureus CPHL GISA | Van/Tec* | 7.8 | 15.6 | 75.37 |
| HO41340156 | (intermediate) | | | (±2.14) |
| S. saprophyticus | ND | 3.9 | 7.8 | 41.67 |
| NCTC 7292 | | | | (±1.13) |
| S. epidermidis NCTC | ND | 3.9 | 7.8 | 57.34 |
| 11047 | | | | (±0.83) |

[Abbreviations: American Type Culture Collection (ATCC), Central Public Health Laboratory UK (CPHL), National Collection of Type Cultures (NCTC), Methicillin (Met), Vancomycin (Van), Teicoplanin (Tec), not determined (ND), epidemic methicillin resistant S. aureus (EMRSA), vancomycin intermediate S. aureus (VISA), gylcopeptide resistant S. aureus (GISA).]
*Other uncharacterised antibiotic resistances may be present.

Table 3 shows that the hydroquinone is active against a range of different staphylococcal strains, indicating its utility either to treat or to prevent staphylococcal infections. These results are likely to be of particular clinical value for the antibiotic resistant test strains.

### EXAMPLE 3

### Activity Against P. acnes NCTC 737

The following experiments all used P. acnes NCTC 737 as the test organism.

MIC, MBC and DDA assays, as described above, were carried out using a range of different benzoquinones and hydroquinones. Supplemented DDA assays, in the presence of salt and lipid, were also conducted.

For most DDA assays, 200 μg of each compound was loaded onto each disc. The exceptions were the assays for 2,6-dimethoxy-p-benzoquinone, in which only 100 μg of the benzoquinone was used. The solvents used were ethanol (for TBHQ, thymoquinone, p-benzoquinone and thymohydroquinone) and DMSO for all other test compounds.

The MIC and MBC results are shown in Table 4 below and the DDA results in Table 5. All results are collated from a number of experiments.

TABLE 4

| | MIC | MBC | MIC/MBC |
|---|---|---|---|
| Test compound | (μg/ml) | (μg/ml) | ratio |
| TBHQ | 7.8 | 15.6 | 0.5 |
| 2,5-dimethyl resorcinol | 3.9 | 31.25 | 0.125 |
| 2,5-dibromo-6-isopropyl-3-methyl-p- | 0.98 | 3.9 | 0.25 |
| benzoquinone | | | |
| 2,6-dimethyl-p-hydroquinone | 7.8 | 31.25 | 0.25 |
| 3,5-di-t-butylcatechol | 7.8 | 15.6 | 0.5 |
| 2,3-dimethyl-p-hydroquinone | 7.8 | 15.6 | 0.5 |
| 2-ethyl-p-hydroquinone | 7.8 | 31.25 | 0.25 |
| 4,6-di-t-butylresorcinol | 7.8 | 15.6 | 0.5 |
| 2-t-butyl-p-benzoquinone | 7.8 | 15.6 | 0.5 |
| 2,3-difluoro-p-hydroquinone | 7.8 | 31.25 | 0.25 |
| 1-o-hexyl-2,3,5-trimethyl-p-hydroquinone | 15.6 | 31.25 | 0.5 |
| thymoquinone | 15.6 | 31.25 | 0.5 |
| 3,5-di-t-butyl-o-benzoquinone | 15.6 | 15.6 | 1 |
| 2,3-dimethoxy-5-methyl-p-benzoquinone | 15.6 | 31.25 | 0.5 |
| 2,5-dimethyl-p-benzoquinone | 15.6 | 31.25 | 0.5 |
| 2,6-dimethoxy-p-benzoquinone | 15.6 | 31.25 | 0.5 |
| 2-methyl-p-benzoquinone | 15.6 | 31.25 | 0.5 |
| 2-chloro-5-methyl-p-benzoquinone | 15.6 | 15.6 | 1 |
| thymohydroquinone | 31.25 | 31.25 | 1 |
| tetrachloro-p-benzoquinone | 31.25 | 62.5 | 0.5 |
| tetrachloro-p-hydroquinone | 31.25 | 31.25 | 1 |
| 2-methyl-p-hydroquinone | 62.5 | 125 | 0.5 |
| 2,5-dichloro-p-hydroquinone | 62.5 | 62.5 | 1 |
| p-benzoquinone | 62.5 | 62.5 | 1 |
| 2-chloro-p-benzoquinone | 62.5 | 125 | 0.5 |
| tetrafluoro-p-hydroquinone | 62.5 | 125 | 0.5 |
| 2-nitroresorcinol | 125 | >250 | <0.5 |
| tetrafluoro-p-benzoquinone | 125 | 250 | 0.5 |

TABLE 5

| | | DDA + | DDA + |
|---|---|---|---|
| | DDA | salt | lipid |
| Test compound | (mm) | (mm) | (mm) |
| TBHQ | 9.95 | 19.48 | 10.37 |
| | (±0.31) | | |
| 2,5-dimethyl resorcinol | 0.0 | 0.0 | 0.0 |
| | (±0.0) | | |
| 2,5-dibromo-6-isopropyl-3-methyl-p | >90 | 71.25 | 34.69 |
| benzoquinone | (±0.0) | | |
| 2,6-dimethyl-p-hydroquinone | 0.0 | 0.0 | 0.0 |
| | (±0.0) | | |
| 3,5-di-t-butylcatechol | 35.93 | 37.26 | 0.0 |
| | (±1.87) | | |
| 2,3-dimethyl-p-hydroquinone | 18.75 | 14.38 | 13.75 |
| | (±1.65) | | |
| 2-ethyl-p-hydroquinone | 9.90 | 9.38 | 0.0 |
| | (±0.36) | | |
| 4,6-di-t-butylresorcinol | 36.59 | 37.83 | 0.0 |
| | (±0.93) | | |
| 2-t-butyl-p-benzoquinone | 60.80 | >90 | >90 |
| | (±2.89) | | |
| 2,3-difluoro-p-hydroquinone | 0.0 | 0.0 | 0.0 |
| | (±0.0) | | |
| 2,5-dimethyl-p-benzoquinone | 44.43 | >90 | 69.28 |
| | (±1.64) | | |
| 1-o-hexyl-2,3,5-trimethyl-p-hydroquinone | 16.64 | 16.43 | 0.0 |
| | (±0.18) | | |
| thymoquinone | 27.46 | 46.49 | 49.01 |
| | (±1.18) | | |
| 3,5-di-t-butyl-o-benzoquinone | 31.83 | 40.52 | 26.39 |
| | (±0.36) | | |
| 2,3-dimethoxy-5-methyl-p-benzoquinone. | 33.40 | 43.35 | 43.98 |
| | (±0.65) | | |
| 2,5-dimethyl-p-benzoquinone | 42.13 | >90 | >90 |
| | (±1.18) | | |
| 2,6-dimethoxy-p-benzoquinone | 0.0 | 16.50 | 15.56 |
| | (±0.0) | | |
| 2-methyl-p-benzoquinone | 35.90 | 42.65 | 48.87 |
| | (±0.36) | | |
| 2-chloro-5-methyl-p-benzoquinone | 38.85 | 37.19 | 38.75 |
| | (±0.65) | | |
| thymohydroquinone | 9.82 | 9.61 | 0.0 |
| | (±0.18) | | |
| tetrachloro-p-benzoquinone | 11.60 | 18.95 | 12.74 |
| | (±0.18) | | |
| tetrachloro-p-hydroquinone | 25.37 | 28.27 | 0.0 |
| | (±1.00) | | |
| 2-methyl-p-hydroquinone | 0.0 | 0.0 | 13.07 |
| | (±0.0) | | |
| 2,5-dichloro-p-hydroquinone | 13.46 | 18.64 | 0.0 |
| | (±0.18) | | |
| p-benzoquinone | 24.87 | 31.44 | 37.98 |
| | (±0.82) | | |
| 2-chloro-p-benzoquinone | 25.79 | 31.69 | 31.38 |
| | (±0.62) | | |
| tetrafluoro-p-hydroquinone | 33.86 | 36.35 | 0.0 |
| | (±1.12) | | |
| 2-nitroresorcinol | 0.0 | 12.71 | 0.0 |
| | (±0.0) | | |
| tetrafluoro-p-benzoquinone | 25.94 | 32.37 | 30.82 |
| | (±0.18) | | |

The data in Tables 4 and 5 show that each of the quinones is active against P. acnes NCTC 737, some strongly so (in particular TBHQ, 2,5-dibromo-6-isopropyl-3-methyl-p-benzoquinone, 3,5-di-t-butylcatechol, 2,3-dimethyl-p-hydroquinone, 4,6-di-t-butylresorcinol, 2-t-butyl-p-benzoquinone, 2,5-dimethyl-p-benzoquinone, 3,5-di-t-butyl-o-benzoquinone, 2,3-dimethoxy-5-methyl-p-benzoquinone, 2,5-dimethyl-p-benzoquinone, 2-methyl-p-benzoquinone, 2-chloro-5-methyl-p-benzoquinone, tetrachloro-p-hydroquinone, p-benzoquinone, 2-chloro-p-benzoquinone, tetrafluoro-p-hydroquinone, tetrafluoro-p-benzoquinone, 2-t-butyl-p-benzoquinone and 2-ethyl-p-hydroquinone—these have a MIC of ≦7.8 μg/ml or a zone diameter of >30 mm on unsupplemented Wilkins-Chalgren agar). In most cases this activity is maintained, at least to some extent, in the presence of salt and lipid, which are important constituents of the human skin environment.

Certain of the benzo/hydroquinones appear to be potentiated by (i.e., their antimicrobial activity increases in the presence of) lipid—these include 2-t-butyl-p-benzoquinone, 2,5-dimethyl-p-benzoquinone, thymoquinone, 2,3-dimethoxy-5-methyl-p-benzoquinone, 2-methyl-p-benzoquinone, p-benzoquinone, 2-chloro-p-benzoquinone and tetrafluoro-p-benzoquinone. Such compounds are likely to be of particular use in the treatment of skin and skin structure conditions, in particular conditions such as acne which can increase skin lipid levels.

### EXAMPLE 4

### Activity Against Other Propionibacteria

The activity of TBHQ was tested against other Propionibacterium spp strains, including some with known antibiotic resistance. MIC, MBC and DDA assays were carried out as described above for each of the strains.

For the DDA assays, 200 μg of TBHQ was loaded onto each disc. The solvent used for the TBHQ was ethanol.

The results are shown in Table 6 below; all are collated from a number of experiments. Table 6 also indicates the resistance phenotype for each of the test strains.

TABLE 6

| | Resistance | MIC | MBC | DDA |
|---|---|---|---|---|
| Test organism | phenotype | (μg/ml) | (μg/ml) | (mm) |
| P. acnes NCTC 737 | None | 7.8 | 15.6 | 8.65 |
| | | | | (±0.31) |
| P. granulosum NCTC | None | 3.9 | 7.8 | 11.00 |
| 11865 | | | | (±0.00) |
| P. acnes PRP-002 | Tet/MLS | 3.9 | 7.8 | 28.42 |
| | | | | (±0.95) |
| P. acnes PRP-003 | Tet | 7.8 | 7.8 | 38.01 |
| | | | | (±1.02) |
| P. acnes PRP-004 | Tet | 1.95 | 7.8 | 30.32 |
| | | | | (±1.97) |
| P. granulosum PRP-005 | MLSK | 62.5 | 62.5 | 0.00 |
| | | | | (±0.00) |
| P. granulosum PPR-006 | MLS | 7.8 | 7.8 | 20.00 |
| | | | | (±2.06) |
| P. acnes PPR-007 | Clin | 3.9 | 7.8 | 13.43 |
| | | | | (±2.49) |
| P. acnes PRP-008 | Clin | 3.9 | 7.8 | 14.47 |
| | | | | (±0.90) |
| P. acnes PRP-010 | MLSK | 3.9 | 15.6 | 18.71 |
| | | | | (±0.18) |
| P. acnes PRP-017 | MLS | 3.9 | 7.8 | 18.40 |
| | | | | (±1.08) |
| P. acnes PRP-023 | MLSK | 3.9 | 7.8 | 23.90 |
| | | | | (±0.00) |
| P. acnes PRP-026 | MLS | 3.9 | 7.8 | 8.18 |
| | | | | (±0.72) |
| P. granulosum PRP-043 | MLS | 15.6 | 15.6 | 10.56 |
| | | | | (±0.48) |
| P. granulosum PRP-044 | MLS | 15.6 | 31.25 | 10.70 |
| | | | | (±0.65) |
| P. acnes PRP-046 | None | 1.95 | 7.8 | 17.46 |
| | | | | (±1.58) |
| P. acnes PRP-053 | Tet/MLS | 3.9 | 7.8 | 23.49 |
| | | | | (±1.44) |
| P. granulosum PRP-055 | None | 3.9 | 7.8 | 13.07 |
| | | | | (±0.78) |
| P. acnes PRP-059 | MLS | 3.9 | 7.8 | 17.56 |
| | | | | (±0.72) |
| P. acnes PRP-068 | Ery | 3.9 | 7.8 | 20.89 |
| | | | | (±1.08) |
| P. acnes PRP-101 | Tet/MLS | 3.9 | 7.8 | 0.0 |
| | | | | (±0.00) |
| P. acnes PRP-102 | Tet/MLS | 7.8 | 15.6 | 23.80 |
| | | | | (±0.10) |
| P. avidum ATCC 25577 | None | 3.9 | 3.9 | 9.16 |
| | | | | (±0.48) |

[Abbreviations: American Type Culture Collection (ATCC), National Collection of Type Cultures (NCTC), Propionibacterium Panel Number (PRP), Tetracycline (Tet), Erythromycin (Ery), Clindamycin (Clin), Macrolide-Lincosamide-Streptogramin (MLS), Macroliode-Lincosamide-Streptogramin-Ketolide (MLSK).]

Table 6 shows that the hydroquinone is active against a range of different propionibacterial strains. This indicates its utility either to treat or to prevent infections associated with such bacteria, in particular acne. The results are likely to be of particular clinical value for the antibiotic resistant test strains.

### EXAMPLE 5

### Activity Against Other Organisms

The activity of TBHQ against three other bacterial strains was investigated using MIC, MBC and DDA assays as described above. For the DDA assays, 200 μg of TBHQ was loaded onto each disc, using ethanol as the solvent.

The results are shown in Table 7 below.

TABLE 7

| | MIC | MBC | MIC/MBC | DDA |
|---|---|---|---|---|
| Test organism | (μg/ml) | (μg/ml) | ratio | (mm) |
| H. influenzae ATCC 49247 | 15.6 | 15.6 | 1 | 36.33 |
| | | | | (±2.31) |
| E. faecalis ATCC 29212 | 15.6 | 31.25 | 0.5 | 33.67 |
| | | | | (±0.58) |
| S. pyogenes ATCC 12344 | 62.5-125 | 62.5-125 | 1 | 50.67 |
| | | | | (±0.58) |

Table 7 shows that TBHQ may be used as an antibacterial agent against species other than staphylococci and propionibacteria, in particular against other Gram-positive cocci.

### EXAMPLE 6

### Topical Anti-Acne Formulations

The results from Examples 1 to 4 show that a benzoquinone or hydroquinone can be an effective antimicrobial agent against staphylococci and propionibacteria. Retention of activity even in the presence of salt, lipid and/or serum also indicates the suitability of such compounds for topical application, in particular to the skin. This can therefore be of use in preparing topical antimicrobial formulations for either prophylactic or therapeutic use in any context where staphylococci and/or propionibacteria are thought to be involved as possible sources of infection.

A topical formulation for use in treating acne may for example be made by formulating a benzoquinone or hydroquinone, in particular an alkyl-substituted benzo/hydroquinone such as TBHQ, in a suitable fluid vehicle, optionally together with conventional additives. Such vehicles and additives may be for instance as found in Williams' “Transdermal and Topical Drug Delivery”, Pharmaceutical Press, 2003 and other similar reference books, and/or in Rolland A et al, “Site-specific drug delivery to pilosebaceous structures using polymeric microspheres”, Pharm. Res. 1993; 10: 1738-44; Mordon S et al, “Site-specific methylene blue delivery to pilosebaceous structures using highly porous nylon microspheres: an experimental evaluation”, Lasers Surg. Med. 2003; 33: 119-25; and Alvarez-Roman R et al, “Skin penetration and distribution of polymeric nanoparticles”, J. Controlled Release 2004; 99: 53-62.

The formulation may be prepared and administered using known techniques. It may for example take the form of a cream, lotion or gel. It may be applied to infected areas of the skin, and/or to areas susceptible to future infection, with a frequency dependent on the nature and severity of the condition and the concentration of the quinone and any other active agents in the formulation, for instance on a daily or twice daily basis.

The concentration of the benzo/hydroquinone may be in the ranges described above, and will be determined based on the intended use of the formulation, its intended mode of administration and the activity of the particular quinone chosen.

### EXAMPLE 7

### Topical Formulation for Use Against Staphylococcal Infections

A formulation for use against S. aureus or other staphylococci may be prepared by formulating a benzo/hydroquinone such as TBHQ in a similar manner to that described for the anti-acne formulation. The ingredients may in this case be formulated as a spray, for instance for application to work surfaces or surgical instruments; as a cleansing gel or lotion for instance for hand washing; as a nasal spray for application to the anterior nares or in many other appropriate forms. Such a formulation may in particular be used prophylactically, e.g., to reduce the risk of outbreaks of MRSA or similar infections.

1-46. (canceled)

47. Method for the treatment, in a human or animal patient, of a condition which is either caused, exacerbated or transmitted by bacterial activity, the method involving the topical application to the patient of a pharmaceutically or veterinarily effective amount of a para-benzoquinone or para-hydroquinone which is substituted with one or more groups selected from alkyl, alkoxy, halogen, hydroxyl, nitro (—NO₂) and amine (—NR₂, where each R is independently either hydrogen or hydrocarbyl) groups attached to carbon atoms in the cyclohexadiene ring.

48. Method according to claim 47, wherein the condition is either caused, exacerbated or transmitted by staphylococcal and/or propionibacterial activity.

49. Method according to claim 47, wherein the condition is a skin or skin structure condition.

50-54. (canceled)

55. Method according to claim 47, wherein the condition is selected from acne, infected atopic eczema, superficial infected traumatic lesions, wounds, burns, ulcers, folliculitis, mycoses and superficial primary and secondary skin and skin structure infections.

56. Method according to claim 55, wherein the condition is acne.

57. Method according to claim 48, wherein the condition is a staphylococcal infection.

58. Method according to claim 57, wherein the benzo/hydroquinone is used prophylactically against staphylococci.

59. Method according to claim 47, wherein the benzo/hydroquinone is administered topically to the skin.

60. Method according to claim 47, wherein the benzo/hydroquinone is administered topically to the nares.

61. Method according to claim 47, wherein the substituents on the benzo/hydroquinone are selected from alkyl groups.

62. Method according to claim 47, wherein the benzo/hydroquinone is mono- or di-substituted.

63. Method according to claim 61, wherein the benzo/hydroquinone is TBHQ.

64. Method for controlling the growth of a staphylococcal bacterium or Propionibacterium, the method comprising applying, to an area infected or suspected to be infected or capable of becoming infected with the bacterium, a para-benzoquinone or para-hydroquinone which is substituted with one or more groups selected from alkyl, alkoxy, halogen, hydroxyl, nitro (—NO₂) and amine (—NR₂, where each R is independently either hydrogen or hydrocarbyl) groups attached to carbon atoms in the cyclohexadiene ring.

65. Method according to claim 64, wherein the benzo/hydroquinone is applied to a non-living surface.
